(19) United States
(12) Patent Application Publication
(10) Pub. No.: US 2024/0084371 A1
(43) Pub. Date: Mar. 14, 2024

(54) SYSTEM FOR EARLY WARNINGS OF CYANOTOXIN PRODUCTION IN SOURCE WATER

(71) Applicant: GOVERNMENT OF THE U.S. AS REPRESENTED BY THE ADMINISTOR OF THE U.S. ENVIRONMENTAL PROTECTION AGENCY, Washington, DC (US)

(72) Inventor: Jingrang Lu, Mason, OH (US)

(73) Assignee: GOVERNMENT OF THE U.S AS REPRESENTED BY THE ADMINISTOR OF THE U.S. ENVIRONMENTAL PROTECTION AGENCY, WASHINGTON, DC (US)

(21) Appl. No.: 17/847,043

(22) Filed: Jun. 22, 2022

(51) Int. Cl. C12Q 1/686; G16B 20/20; G16B 25/10

(52) U.S. Cl. CPC C12Q 1/686; G16B 20/20; G16B 25/10

(57) ABSTRACT

A computer system for generating early public warnings and predictions of cyanotoxin production in source water comprised of the a processor for instantiating RT-qPCR test data objects for storing RT-qPCR gene expression data wherein each RT-qPCR test data object is identified by test location, test year. Each RT-qPCR test data object includes one or more multi-dimensional array objects. Each multi-dimensional array object is configured to store ordered sets of data wherein each of said ordered pairs is comprised of measurement dates and a detection value.

### STATEMENT REGARDING FEDERALLY SPONSORED RESEARCH OR DEVELOPMENT

The invention described herein was made by an employee of the United States Government and may be manufactured and used by the Government of the United States of America for governmental purposes without the payment of any royalties. This and related patents are available for licensing to qualified licensees. Please contact Carmen Krieger at 202.564.0396 for more information.

### CLAIM OF PRIORITY

This application claims priority to U.S. application Ser. No. 16/142,319 filed on Aug. 29, 2018

### FIELD OF INVENTION

The present invention relates to a an early alert system for detecting and communicating information pertaining to water safety using assays disclosed in application Ser. No. 16/142,319 which has the capability to simultaneously conducting testing for a plurality of cyanobacteria which carry a gene to produce cyanotoxins, using standardized test conditions.

### BACKGROUND OF THE INVENTION

The U.S. Environmental Protection Agency (EPA) publishes an annual list of the top thirty unregulated contaminants that are known or expected to occur in public water systems in the U.S. Ten of the thirty contaminants of concern are toxins produced by a common type of bacteria called cyanobacteria.

Cyanobacteria, also called blue-green algae, are microscopic organisms found naturally in all types of water. A “cyanobacteria bloom” is an event during which cyanobacteria multiply very quickly. Blooms can form in warm, slow-moving waters that are rich in nutrients from fertilizer runoff or septic tank overflows, and most often occur in summer or early fall.

Toxic or harmful cyanobacteria blooms are events in which the concentration of cyanotoxins in a water supply exceed levels deemed safe for humans and other species. Cyanotoxins have been associated with minor symptoms such as rashes, and also more serious liver and brain damage.

Most cyanobacteria blooms do not produce toxins at a sufficient level to compromise public water supplies and cause harm to humans and other species. The vast majority of cyanobacteria species do not produce toxins. However, toxic species of cyanobacteria produce multiple types of toxins during a bloom. The aggregate level of all types of cyanotoxins produced by all species known to be carriers may cause the toxin level to exceed a safe threshold for humans and other species.

Historically, water supplies have been monitored by measuring cyanobacteria count and biomass to determine the presence of cyanobacterial species and their blooms, without differentiating species that carry harmful toxin genes or the genotypes of toxins produced. Public concern over cyanobacterial blooms has increased due to their higher frequency of occurrence and their potential ecological, economic and health impacts. U.S. patent application Ser. No. 16/142,319 teaches the election of microcystin (MC) producers (MCPs) using qPCR and RT-qPCR, allowing for the rapid identification of blooms by combining specificity and sensitivity with a relatively high throughput capability. U.S. patent application Ser. No. 16/142,319

U.S. patent application Ser. No. 16/142,319 teaches the Investigation of MCP population composition (correlation, dominance), toxin gene expression, and relationship to MC concentration was conducted using a panel of qPCR assays targeting mcyA, E and G on weekly and daily water samples collected from an Ohio inland reservoir lake.

Data derived from these assays were used to develop early warning thresholds for prediction of MC concentrations exceeding the US EPA Health Advisory cutoff value (>0.3 μg L⁻¹) using receiver operating characteristic curves and tobit regression.

In one study, MCP Microcystis genomic copy number made up approximately 35% of the total Microcystis spp. and was the dominant toxic subpopulation of MCPs. Microcystis toxin genes increased in June and July but decreased in August and September along with similar trends of cell replication. Quantities of both RT-qPCR and qPCR followed the same trend and were highly correlated with MC-ADDA, while RT-qPCR not only reflected the active toxin genes or toxic species, but also indicated the beginning and ending of toxin production

In the foregoing study, a one-week early warning of MC exceedance over the EPA Health Advisory was based on signaling of qPCR and RT-qPCR using receiver operating characteristic curves. This study illustrates the potential use of qPCR or RT-qPCR as an early warning system of extant and MC producing potentials during a toxic algal bloom, with predictive high powers.

More recently, assays have been developed to perform quantitative polymerase chain reaction (qPCR) and reverse transcription qPCR (RT-qPCR) methods known in the art. These test methods known in the art can detect the presence of a single toxin gene type, across multiple species. The number of gene copies detected can be correlated to future levels for the individual toxin.

There is a further unmet need for a public alert and communication system implemented with software and a system having control logic that can be coupled with data derived from the assay to assist water treatment personnel, government and health officials and policy makers in quickly interpreting quantitative data from the assay to efficiently alert the public as to events, predicting future events and determining the efficacy of remediation methods

### SUMMARY OF THE INVENTION

A computer system for generating early public warnings and predictions of cyanotoxin production in source water comprised of the a processor for instantiating RT-qPCR test data objects for storing RT-qPCR gene expression data wherein each RT-qPCR test data object is identified by test location and dates. Each RT-qPCR test data objects includes one or more multi-dimensional arrays objects. Each multi-dimensional array objects is configured to store ordered pairs of data wherein each of said ordered pairs is comprised of measurement dates and a detection value.

The system generates an alert when one of said detection values exceeds user determined threshold values. In various embodiments, the user defined value may be higher than zero, or a valued determined reflecting the potential level of cyanotoxin production that exceeds EPA Guidelines

In various embodiments, the system performs a trend function compares the detection values of the gene copy numbers from current and past sample dates. A trend state is calculated based on detection values of the number of cyanotoxin gene copies. A trend state may be described as increasing, peak, decreasing and end (FIG. 1).

In various embodiments, the trend processor is configured to performs said trend function using a moving average calculation and a comparison operation as to the number of gene copies present. In still other embodiments, the trend processor performs a trend direction function calculation to determine the rate at which said MC detection values are changing.

In various embodiments, the system may be configured to predict cyanotoxin level estimated based on the correlations between qPCR-based gene copies and ELISA-based cyanotoxin concentrations and to compare the estimated cyanotoxin concentration level can be compared with EPA guideline level to indicate alert.

In still other embodiments, the system may be configured to perform a probability function on one or more on said ordered pairs within one or more qPCR test data objects for a current year and to perform a calculation to determine the probability of types, level and duration of cyanotoxin production according to modeling calculation based on previous year datasets and current year's water parameters.

### TERMS OF ART

As used herein, the term, “aggregate number of gene copies” means the total number of gene copies present in a sample for the four toxins tested which contribute to overall toxin levels.

As used herein, the term, “early alert” means that an one approximate week alert or warning for cyanotoxin production will be given, when a certain level of RT-qPCR signal is detected before a cycle of cyanobacterial bloom starts.

As used herein, the term, “comparable test results” means test data which is obtained under standardized test conditions so that it is mathematically comparable and may be aggregated and analyzed relative to multiple toxin types.

As used herein, the term “detection value” means a value that is detected above detection threshold.

As used herein, the term “gene expression” is the targeted gene transcripts determined by RT-qPCR. The targeted gene can be any gene selected from a group consisting of production of microcystin, anatoxin, saxitoxin, and cylindrospermopsin.

As used herein, the term, “standardized test conditions” means a qPCR or RT-qPCR running condition according to a SOP.

As used herein, the term, “rate of change” means the change of the toxin-producing gene copy numbers between successive sampling points calculated from a simple first-order rate law using the equation.

As used herein, the term, “trend status” means a status characterizing the rate of change as in a state of increase, peak, stagnant, decrease and end.

As used herein, the term “predictive modeling” means specific modeling for a single location on historical pattern, current water parameters to predict the occurrence, intensity and duration of current year.

As used here in “processor means” a virtual compute processing component which performs a specific computational function defined by a software method which, draws upon the general capability of the program when invoked.

As used herein, the term “gene copy” means the number of copies of a particular gene in the genotype of an individual

As used herein, the term “data object” means a reusable software object which may be configured or instantiated with both executable code and data values.

As used herein, the term “PCR” is the abbreviation of polymerase chain reaction employing primers and a DNA polymerase. The primers used are the cyanotoxin-specific or toxic-species specific oligo DNA fragments as are shown in the previously submitted patent.

As used herein, the term “qPCR” means quantitative polymerase chain reaction. A serial of known gene quantity will be used as standard and the unit of quantity of a filtered water sample is copy number L⁻¹. A used herein, the term “RT-qPCR” indicates a reverse transcription polymerase chain reaction. In RT-qPCR, total RNA isolated from filtered water samples will be firstly transcribed to DNA (denoted as cDNA), and then regular qPCR will be conducted using the same primers as in qPCR, while the resulted quantity is mRNA copy number with the unit copy number L⁻¹.

As used herein, the term “CTP” indicates cyanotoxin production.

### BRIEF DESCRIPTION OF THE DRAWINGS

FIG. 1 is a flow chart which illustrates an exemplary methods for generating an early warning alert (Method 100) to advise the public of a potentially hazardous level of cyanotoxin production in the future and for monitoring the status (Method 200) of a cyanotoxin production (CTP) event.

FIG. 2 illustrates an exemplary embodiment of Method 300 for predicting the probably occurrence dates of cyanotoxin production in a year.

FIG. 3 illustrate n exemplary Method 400 for generating alerts based on alternative EPA guidelines Including an alert for a potential hazardous level of cyanotoxin production (CTP)

### DETAILED DESCRIPTION OF THE INVENTION

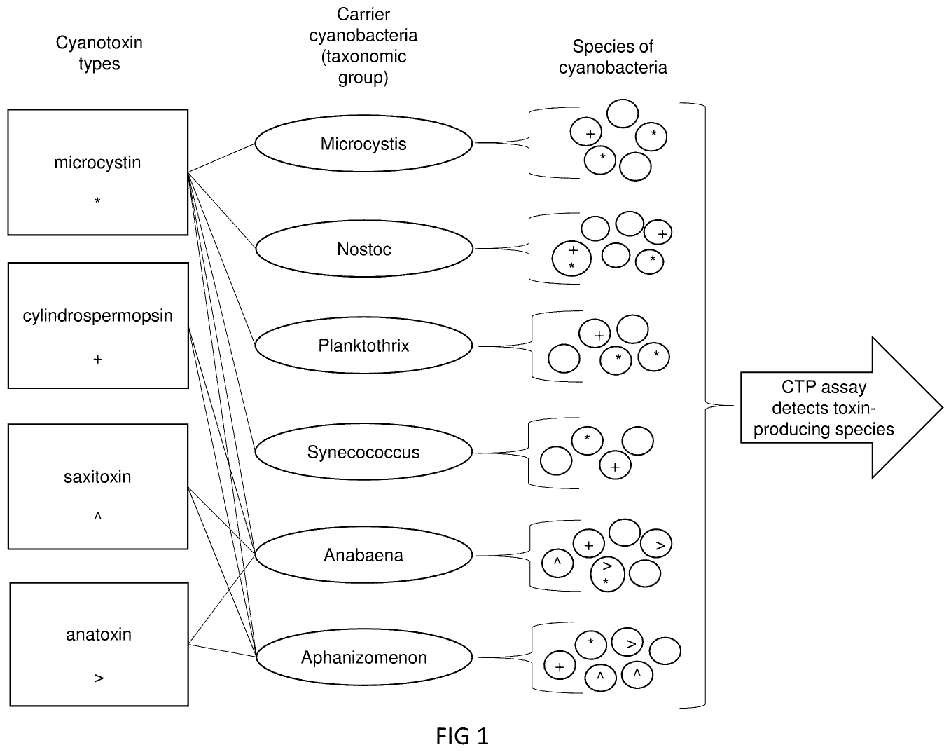

FIG. 1 provides an overview of events which can be warned and detected by RT-qPCR method, indicates that the current status of MC production can be estimated or calculated, and the MC productivities can be at various points in time when RT-qPCR signals are received by the user. In various embodiments, the system provides control logic to send alert and generate reports as to anticipated time frames for [MC≥μg L⁻¹] based on the results of detection using the novel assays. Responsive action includes, but is not limited to, generation of reports and alarm signals that are delivered in near real-time to users of the system.

is a diagram which illustrates how one exemplary embodiment of a Cyanotoxin Prediction (CTP) Assay Panel can be used to more accurately detect the presence of toxin-producing cyanobacteria in a water sample.

FIG. 1 illustrates four known types of cyanotoxins (microcystin, anatoxin, saxitoxin, and cylindrospermopsin) which are produced by more than one subgroup of cyanobacteria, represented as ovals. Each subgroup of cyanobacteria includes multiple species, represented as circles; however, only a few of these species produce cyanotoxins. Species carrying toxic genes that produce cyanotoxins are represented by an asterisk or symbol in the circle.

In the exemplary embodiment shown, the CTP Assay Panel distinguishes between toxic and non-toxic species to specifically detect the presence of toxic species.

The CTP Assay Panel identifies and distinguishes the presence of toxic subgroups of cyanobacteria through the use of novel oligonucleotide primers and quantitative polymerase chain reaction (qPCR) amplification methods known in the art.

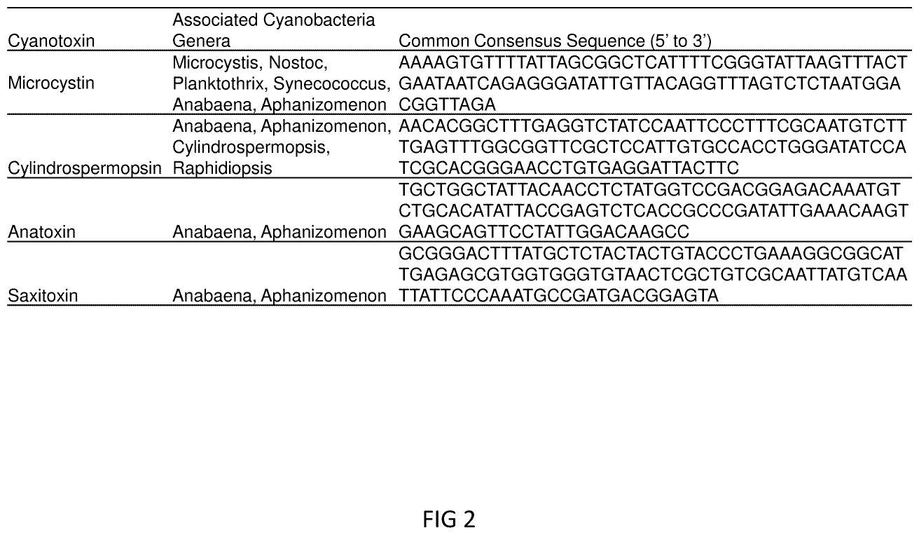

FIG. 2 is a table identifying multiple cyanotoxins, the carrier group of cyanobacteria associated with each cyanotoxin gene, and the common DNA sequences which define a member of the cyanobacteria carrier group.

The right-most column illustrates the common DNA sequences identified by the invention. These sequences are common in multiple species and allow simultaneous testing for four different toxin genes to simultaneously detect the presence of multiple species that produce cyanotoxins.

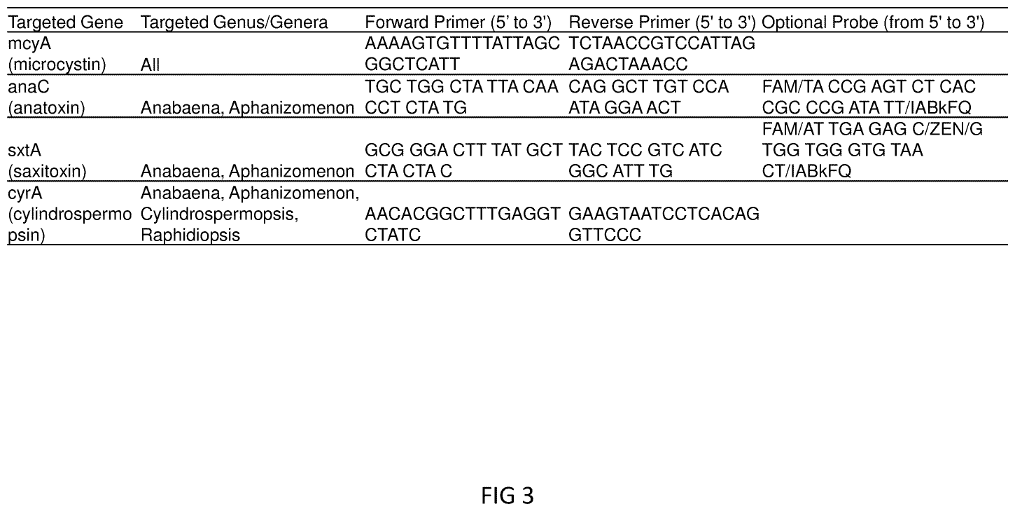

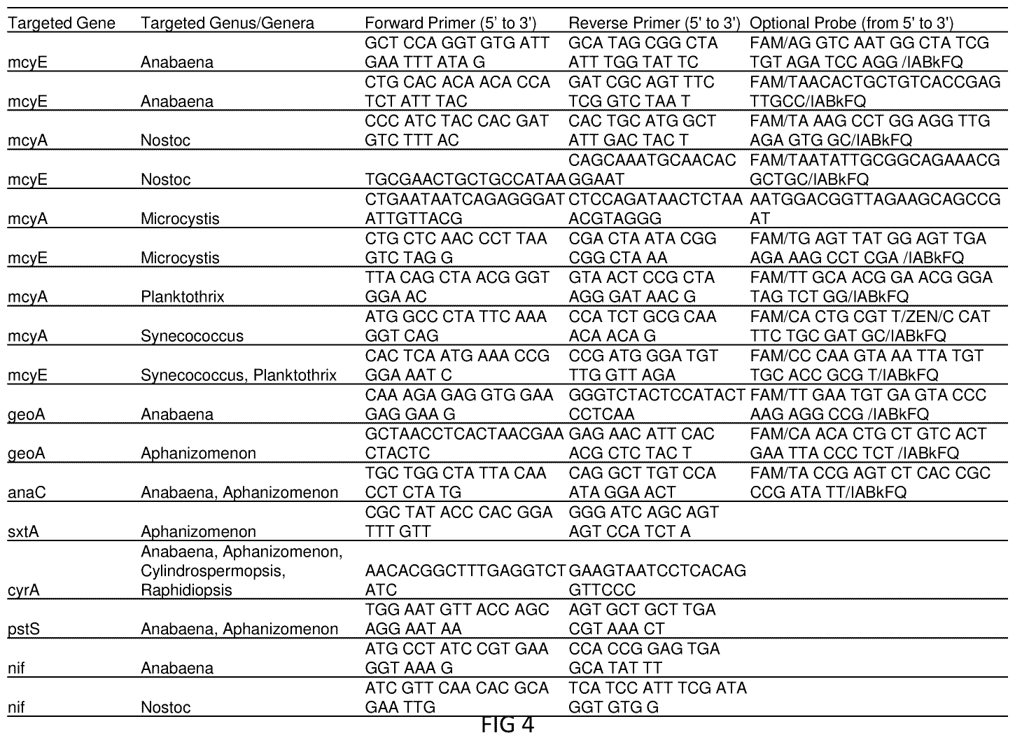

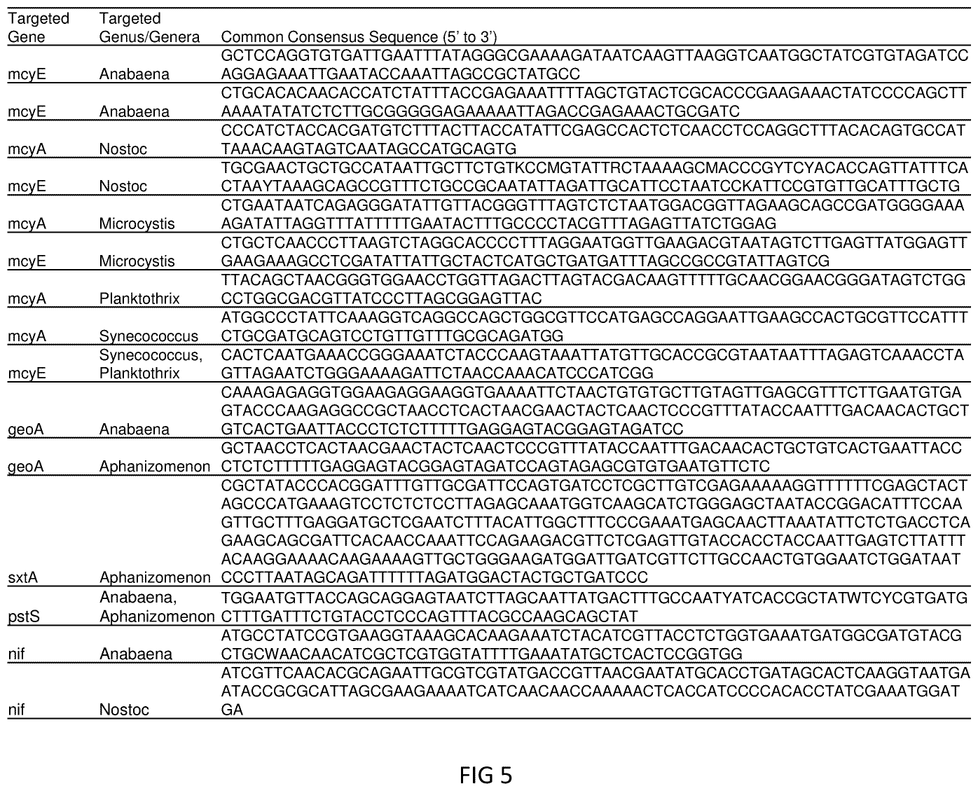

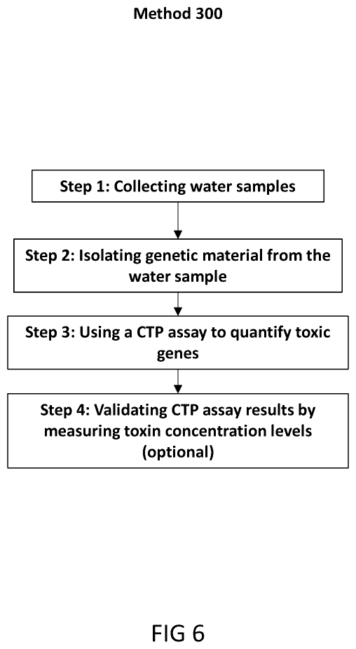

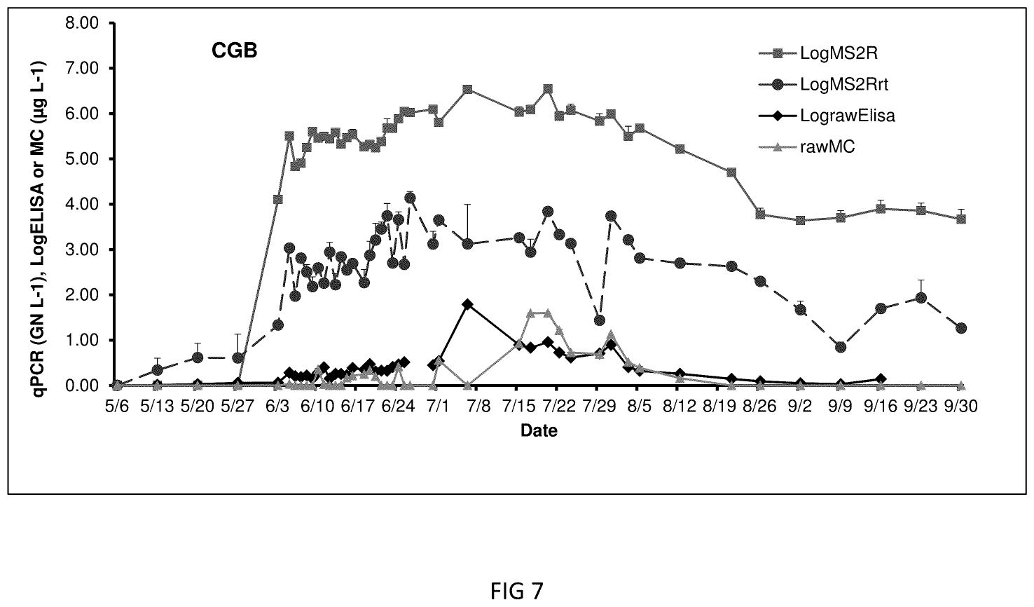

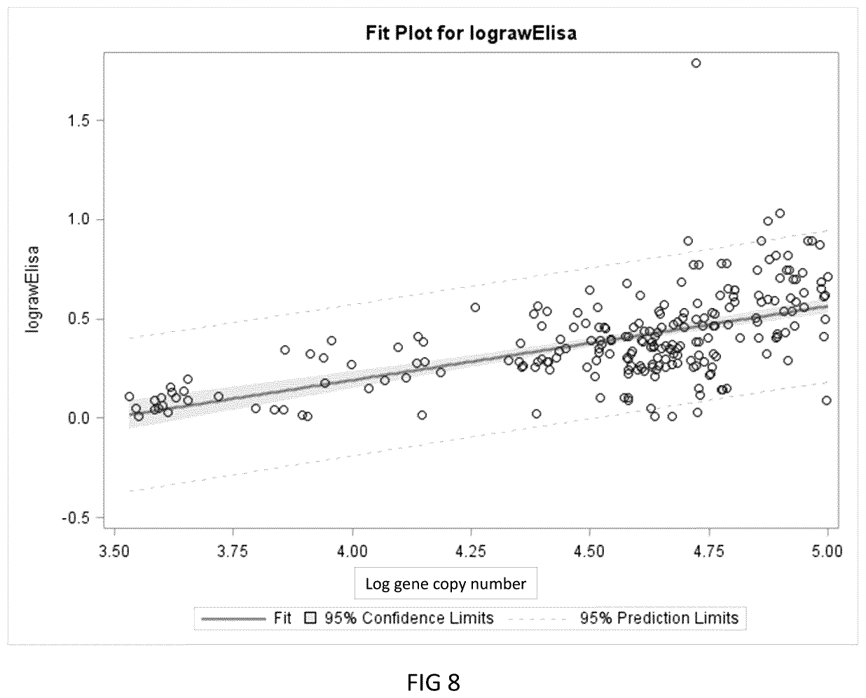

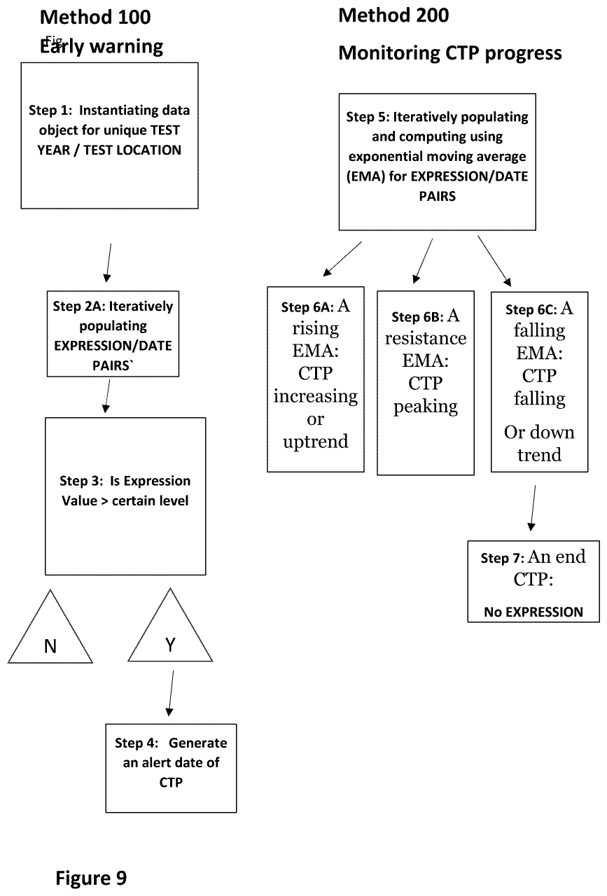

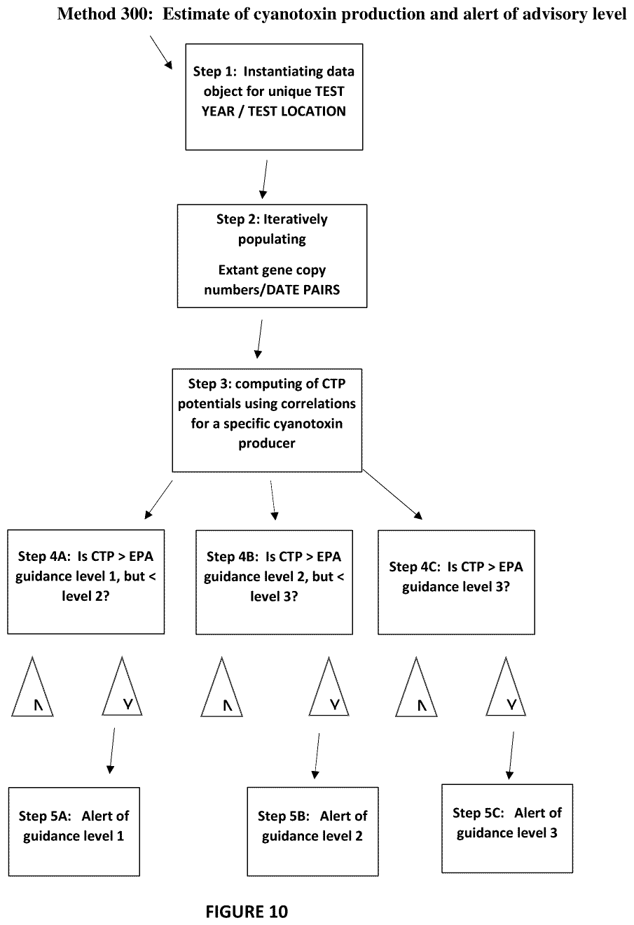

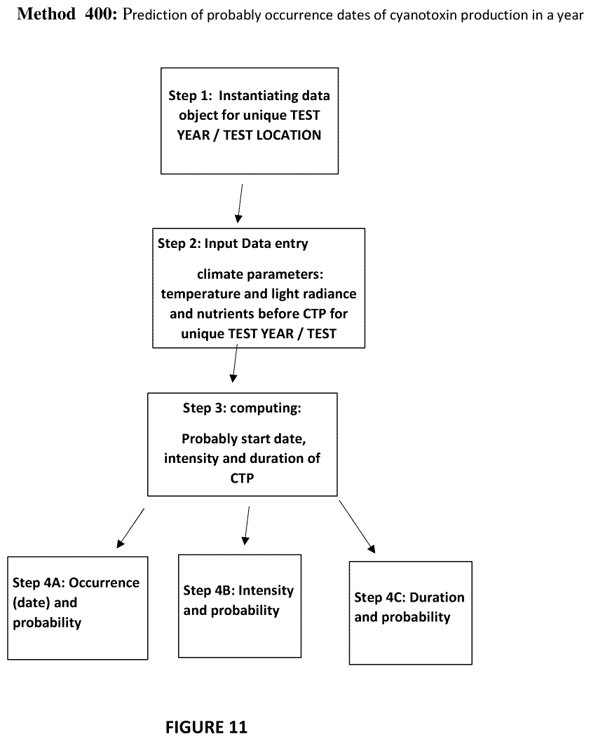

FIG. 3 is a table illustrating exemplary primer and probe sequences which can be used to produce CTP Assay Panel 100 for early detection and warning.

In one exemplary embodiment, CTP Assay Panel 100 is a panel of RT-qPCR/qPCR assays for detecting cyanotoxin genes, which include the novel primer pairs described in FIG. 3. In this embodiment, the primer pairs are designed to detect multiple species of toxic cyanobacteria simultaneously. The primers shown each have a sequence that will bind to a cyanotoxin gene at 60-64 degrees Celsius. In various embodiments, the recommended annealing temperature for the primers shown is 62 degrees Celsius. This common annealing temperature allows all assays to be conducted simultaneously. Without these novel primers and standardized conditions for multiple species, it was not possible to integrate all of the test results for multiple species. Standardized qPCR reaction conditions produce statistically comparable qPCR data from samples with different species, taken from geographically diverse waters.

In this exemplary embodiment, the assays are standardized with the same common annealing temperature, thermocycle duration, and control samples designed to yield consistent qPCR test results. In various embodiments, CTP Assay Panel 100 further includes approximately four to six positive control samples, each having a unique number of cyanotoxin gene copies within a range of approximately 1,000 to 10,000 DNA gene copies per liter.

In one embodiment, simultaneous detection of the mcyE/mcyA, sxtA, cyrA, or anaC genes indicates possible production of microcystin, saxitoxin, cylindrospermopsin or anatoxin, respectively. In this exemplary embodiment, the RT-qPCR/qPCR assay detects the presence of cyanotoxin genes in control samples and collected water samples or other test samples. In various embodiments, CTP Assay 100 can be used to determine the total number of gene copies for each cyanotoxin gene and estimate the population size of each group of toxic cyanobacteria.

In the exemplary embodiment shown, each primer pair selected for qPCR analysis targets a sequence of cyanotoxin biosynthesis genes and genus-specific genes that is common to multiple cyanobacteria species. The target genes encode cyanotoxins, including microcystin, anatoxin, saxitoxin, and cylindrospermopsin. Targeted genes include an mcyA gene sequence carried by cyanobacteria in all six genera, an anaC gene sequence carried by cyanobacteria in the Anabaena and Aphanizomenon genera (exemplary detected species include Aphanizomenon gracile, Anabaena sp., and Anabaena circinalis), an sxtA gene sequence carried by cyanobacteria in the Anabaena and Aphanizomenon genera, and a cyrA gene sequence carried by cyanobacteria in the Anabaena, Aphanizomenon, Cylindrospermopsis, and Raphidiopsis genera (exemplary detected species include Raphidiopsis curvata and Cylindrospermopsis raciborskii).

FIG. 4 is a table illustrating primer and probe sequences which can be used to produce CTP Assay Panel 200 for identifying dominant toxic groups of cyanobacteria.

CTP Assay Panel 200 can detect multiple toxic species simultaneously. In various embodiments, CTP Assay Panel 200 can detect the number of toxic gene copies and predict the level of toxin that will be produced by each type of cyanobacteria individually and in the aggregate.

In an alternative embodiment, CTP Assay Panel 200 is comprised of a panel of multiple RT-qPCR/qPCR assays that include the primers shown in FIG. 4A. In this embodiment, the RT-qPCR/qPCR assay detects the presence or absence of individual toxin-producing subtypes of cyanobacteria to determine dominant toxic groups in control samples and collected water samples or other test samples. This embodiment more specifically determines which individual subtypes of cyanobacteria are present and which has the highest population. Each primer shown has an annealing temperature of approximately 60 to 64 degrees Celsius. In various embodiments, the recommended annealing temperature for the primers shown is 62 degrees Celsius.

In the alternative embodiment, alternative primer pairs can detect an mcyA or mcyE gene sequence carried by cyanobacteria in the Anabaena, Nostoc, Microcystis, Planktothrix, and Synecococcus genera (exemplary detected species include Anabaena sp., Anabaenopsis elenkinii, Anabaena lemmermannii, Anabaena flos-aquae, Nostoc sp., Fischerella sp., Nodularia spumigena, Nodularia sphaerocarpa, Nodularia sp., Microcystis sp., M. aeruginosa, M. viridis, M. panniformis, M. wesenbergii, M. smithii, Planktothrix sp., P. rubescens, P. agardhii, Synechococcus sp., WH 8103, and WH8102), an anaC gene sequence carried by cyanobacteria in the Anabaena, and Aphanizomenon genera, an sxtA gene sequence carried by cyanobacteria in the Aphanizomenon genus, a cyrA gene sequence carried by cyanobacteria in the Anabaena, Aphanizomenon, Cylindrospermopsis, and Raphidiopsis genera (exemplary detected species include Raphidiopsis curvata and Cylindrospermopsis raciborskii), a geoA gene sequence carried by cyanobacteria in the Anabaena and Aphanizomenon genera (exemplary detected species include Dolichospermum ucrainicum, D. planctonicum, D. circinale, Nicotiana attenuate, and Anabaena ucrainica), a pstS phosphase gene sequence carried by cyanobacteria in the Anabaena and Aphanizomenon genera, and a nif gene sequence carried by cyanobacteria in the Anabaena and Nostoc genera.

FIG. 5 illustrates the common genetic sequences which are detected by the primers and probes used to produce CTP Assay Panel 200 for identifying dominant toxic groups of cyanobacteria.

FIG. 5 illustrates the common DNA sequences which define a member of the cyanobacteria carrier group detected by the CTP Assay Panel 200 primers and probes.

FIG. 6 illustrates exemplary Method 300 for using CTP Assay Panel 100 and/or 200 to measure cyanotoxin genes and correlating CTP Assay results to subsequently measured cyanotoxin levels.

In the exemplary embodiment shown, Method 300 utilizes a panel of novel qPCR/RT-qPCR assays for simultaneously detecting microcystin, anatoxin, saxitoxin, and cylindrospermopsin genes in cyanobacteria. The invention is a testing method for detecting specific bacterial groups associated with toxin production.

In various embodiments, Method 300 may be used to identify the number of gene copies present and predict the amount of toxin that will be produced by each cyanobacteria genus individually and in the aggregate. In various embodiments, Method 300 utilizes analysis of the qPCR/RT-qPCR results to predict whether cyanotoxin concentrations in a source of water will be exceed a toxic threshold deemed harmful to humans and other species within a specified period of time. In various embodiments, the toxic threshold is a limit set by U.S. EPA Drinking Water Health Advisories. For example, the threshold for combined microcystin toxins is 0.3 μg/liter and a gene copy number of 1,000 to 10,000 DNA gene copies per liter predicts that the toxic threshold will be exceeded seven days after measuring the gene copy number.

Step 1 is the step of collecting water samples. In various embodiments, this step is accomplished by periodically collecting water samples from the same source, at various points in time.

Step 2 is the step of isolating genetic material from a water sample.

In one exemplary embodiment this step is accomplished by dividing samples 100-300 mL aliquots and individually filtering the aliquots using EMD Millipore Durapore™ membrane filters (0.40 μm, MilliPore, Foster City, CA) for DNA extraction. In one embodiment, DNA and RNA are extracted using a kit known in the art, such as AllPrep DNA (QIAGEN, Valencia, CA). Filtered aliquots are stored at −80° C. in 1.5 mL microtubes with lysis buffer prior to extracting DNA and RNA.

In various embodiments, this step includes using any method known in the art for isolating or extracting genetic material from a water sample and conducting reverse transcription to create template DNA from RNA.

Step 3 is the step of using CTP Assay Panel 100 and/or 200 to determine the number of copies of toxic genes.

To conduct a qPCR/RT-qPCR assay, components are combined and heated to create a polymerase chain reaction. In one exemplary embodiment, each reaction contains 1 μM concentration of each selected primer, 2 μl of template DNA from either the sample or the control, a 0.2 mM concentration of each of the four deoxynucleoside triphosphates (dTTP, dCTP, dGTP, and dATP), 1.5 mM MgCl2, 1 μM (each) primer, and 2.5 U of TaqDNA polymerase (Clone Tech, Mountain View, CA) in a total volume of 25 μl. In various embodiments, the effective primer concentration range for the PCR reaction is approximately 0.5 to 1 μM. In this embodiment, the reactions are heated and cooled during 25 cycles of temperature changes, wherein each cycle includes 1 minute of denaturation at 94° C., 1 minute of primer annealing at 62° C., and 5 minutes of primer extension at 72° C. In various embodiments, the annealing temperature is approximately 60 to 64° C.

In various embodiments, this step further includes analyzing the results by methods known in the art to determine the gene copy number in each sample, for each cyanotoxin gene detected in that sample. In various embodiments, this step may include running CTP Assay Panel 100 on a Juno robot platform where 40 assays can be run at one time, including 1,600 reactions.

Step 4 is the optional step of validating CTP Assay Panel 100 and/or 200 results by measuring toxin concentration levels on a subsequent date using a testing method known in the art and comparing the measured toxin concentration levels to the results of CTP Assay Panel 100.

FIG. 7 summarizes exemplary data reporting the number of toxic gene copies and the concentration of cyanotoxins measured during periodic testing of a water source. FIG. 7 summarizes the number of toxic gene copies in DNA isolated from a water source and measured once per week between May 6 and September 30. Data marked LogMS2R were measured by a quantitative polymerase chain reaction (qPCR) assay (represented by squares) and data marked LogMS2Rrt were measured by a reverse transcription quantitative polymerase chain reaction (RT-qPCR) assay (represented by circles).

In the exemplary embodiment shown, the concentration of cyanotoxins in a water source was measured by an enzyme-linked immunosorbent assay (ELISA), represented by diamonds. The raw concentration of cyanotoxins measured by ELISA is represented by triangles.

The x-axis shows dates and the y-axis shows gene copy number or toxin concentration on a logarithmic scale.

In alternative embodiments, the concentration of cyanotoxins in a water source is measured by liquid chromatography-tandem mass spectrometry (LC-MS/MS).

FIG. 8 summarizes exemplary data correlating the number of toxic gene copies measured by CTP Assay Panel 100 and/or 200 to the subsequent concentration of cyanotoxins measured by an enzyme-linked immunosorbent assay (ELISA).

FIG. 8 shows a regression, which is the best fit curve, of the correlation between the number of toxic gene copies measured by qPCR and RT-qPCR and the concentration of cyanotoxins in a water source measured by an enzyme-linked immunosorbent assay (ELISA). FIG. 8 demonstrates that there is a direct correlation between the expression of the toxic gene and the production of the toxin. The gene copy number is plotted on the x-axis on a logarithmic scale and the associated cyanotoxin concentration measured seven days after the gene copy number from the same water source is plotted on the y-axis on a logarithmic scale. In the exemplary embodiment shown, measured data are represented by open circles. The best fit curve showing a predicted toxin concentration on the y-axis for a given gene copy number measured by CTP Assay Panel 100 or 200 is represented by a dark, thin solid line. The 95% confidence interval of the best fit curve is represented by a thicker, lighter line. The 95% prediction limits showing a range of toxin concentration levels predicted by a given gene copy number are represented as dashed lines.

FIG. 9 is a flow chart which illustrates exemplary Method 100 for generating an early warning of cyanotoxin production in the future and Method 200 for monitoring the trend status of a particular event.

FIG. 9 illustrates Method 100 for generating an early warning using the first detected indicator RT-qPCR signals for CTP, while the Method 200 for monitoring the development of the CTP event including the duration and intensity of a single CTP event.

Step 1 of Methods 100 I and 200 s is the step Instantiating data object for unique TEST YEAR/TEST LOCATION

Step 2 of Methods 100 and 200 is the step Iteratively populating gene copy numbers/DATE PAIRS

Step 3 of Methods 100 and 200 is the step determining the first detected RT-qPCR signals.

Step 4 of Methods 100 and 200 is the step computing the possible date of CTP for a specific cyanotoxin producer.

Step 5 of Methods 100 and 200 is the step Iteratively populating and computing using exponential moving average (EMA) for expression data.

iteratively comparing gene expression signals (detection value: copy number L⁻¹).

In one exemplary embodiment of Method 100, the value in step 4 is higher than certain value, indicating to trigger an alert when a gene expression is first detected. In other embodiments, the value is determined by EPA guidelines

To perform the Methods 100 and 200 illustrated in FIG. 9, a data object is instantiated for storing RT-qPCR test data objects, objects for storing RT-qPCR gene expression data wherein each RT-qPCR test data object is identified by test location, test year.

In the exemplary embodiment shown, each RT-qPCR test data objects include one or more multi-dimensional arrays object, and is configured to store ordered pairs of data wherein each of said ordered pairs is comprised of measurement dates and a detection value indicating the number of gene copies detected.

in various embodiments of the method, an alert can be sent with the detection values exceeds a certain level. In other embodiments, the detection value is continuously updated and compared to a standard, such as EPA Guidelines to determine when initiate an early warning alert.

Various embodiments, Methods 100 and 200 further include the step calculating a trend status based by comparing stored detection values for the current date to detecting values of a prior date.

In one exemplary embodiment using Method 200, the trend function incudes the steps of: (1) calculating moving averages at user defined intervals; (2) comparing the moving average to know if increasing, peak, decreasing; and (3) displaying an alert to reflect a change in trend status.

FIG. 10 illustrate an exemplary Method 300 for generating alerts based on the correlation between gene copy numbers and cyanotoxin concentrations. When a potential cyanotoxin level is estimated, an alert can be generated according to EPA guidelines.

FIG. 11 illustrates an exemplary embodiment of Method 400 for predicting the probability of occurrence dates of cyanotoxin production in a year. In this exemplary embodiment, the method includes the step of performing a probability function on one ordered pairs within one or more qPCR test data objects for a current year to determine the probability of types, level and duration of cyanotoxin production according to modeling calculation based on previous year datasets and current year's water parameters.

In various embodiments, Method 400 may include the step of performing a nonlinear regression analysis function to predict the toxin level. In various embodiments, the nonlinear regression analysis function may predict toxin level with a 95% confidence interval.

In various embodiments, the parameters may reflect water quality present in the designated body of water under test, including but not limited to nutrients (nitrogen and phosphorus and trace elements) and physical parameters (temperature and light, etc).

What is claimed is:

1. A computer system for generating early warnings and predictions of cyanotoxin production in source water comprised of:
initiating RT-qPCR test data objects for storing RT-qPCR gene expression data wherein each RT-qPCR test data object is identified by test location, test year;
wherein each of said RT-qPCR test data objects includes one or more multi-dimensional arrays objects;
wherein each of said multi-dimensional array objects is configured to store ordered pairs of data wherein each of said ordered pairs is comprised of measurement dates and a detection value; and
at least one alert processor which generates an alert when one of said detection values exceeds a certain level.

2. The apparatus of claim 1 wherein each of said detection values is a value that reflects the number of a toxin gene expression level detected on said measurement date.

3. The apparatus of claim 2 wherein said gene expression are copies of a gene selected from a group consisting of production of microcystin, anatoxin, saxitoxin, and cylindrospermopsin

4. The apparatus of claim 1 which further includes at least trend processor configured to perform a trend function wherein said trend function compares the detection values of one or more ordered pairs of gene copy data having current measurement dates to the detection values of the ordered pairs having prior measurement dates to identify a trend state

5. The apparatus of claim 4 wherein said trend state is calculated based on detection values reflecting the number of cyanotoxin gene copies

6. The apparatus of claim 4 wherein said trend state is selected from a group consisting of the following increasing, peak, decreasing and end.

7. The apparatus of claim 4 which includes wherein said alert processor which is configured to perform an alert function and generate an alert when there is a change in the trend state.

8. The apparatus of claim 4, wherein said trend processor is configured to performs said trend function using a moving average calculation and a comparison operation.

9. The apparatus of claim 4 wherein said trend processor performs a trend direction function calculation to determine the rate at which said gene expression values are changing.

10. The apparatus of claim 4 wherein the trend is based on the growth rate of dominant toxin-producing cyanobacteria.

11. The apparatus of claim 4 wherein the trend is base based on a running average of detections values on successive sampling dates.

12. The apparatus of claim 4 wherein said trend processor is configured perform a rate of change function to identify the rate of change of said trend status.

13. The apparatus of claim 12 wherein said trend processor is configured to identify the rate of change of said trend status based on the number of gene copies

14. The apparatus of claim 12 which further includes a graphical interface which is a processor which performs functions to convert data stored in one or more said multi-dimensional array objects into a graphical representation of measurement dates and detection values to graphically illustrate a trend.

15. The apparatus of claim 12 wherein said trend processor is configured to process test user-defined testing intervals.

16. The apparatus of claim 12 wherein the user-defined testing is from seven to ten days

17. The apparatus of claim 1 which further configured to all allow a user to select user-selected parameters and values for generating an alert. elected from a group consisting of a single MC detection value, a trend, a rate, a value obtained from a rolling average calculation

18. The apparatus of claim 17 which is further configured to predict cyanotoxin level estimated based on the correlations between qPCR-based gene copies and ELISA-based cyanotoxin concentrations and to compare the estimated cyanotoxin concentration level can be compared with EPA guideline level to indicate alert.

19. The apparatus of claim 1, which is further configured to perform a probability function on one or more on said ordered pairs within one or more qPCR test data objects for a current year and to perform a calculation to determine the probability of types, level and duration of cyanotoxin production according to modeling calculation based on previous year datasets and current year's water parameters.

20. The apparatus of claim 20 wherein said predictive modeling processor is configured to receive parameters selected from a group consisting of physical parameters and chemical parameters over time.

21. The apparatus of claim 19 wherein said nutrient parameters reflect quantities of nutrients selected from a group consisting of nitrates, phosphates, sulphates and iron.

22. A method for processing qPCR test data and predict cyanotoxin levels for a single body of water comprised of the steps of:
Iteratively extracting the data form qPCR test results;
populating an array to store date and number of gene copies per liter of water;
and generating an alert if the aggregate number of gene copies is greater than a certain level; and identifying the gene as a gene copy from a group consisting of microcystin, anatoxin, saxitoxin, and cylindrospermopsin.

23. The method of claim 23 which performs a nonlinear regression analysis function to predict the toxin level.

24. The method of claim 23 wherein said nonlinear regression analysis function predicts said toxin with a 95% confidence interval
